(19) United States
(12) Patent Application Publication
(10) Pub. No.: US 2009/0227997 A1
(43) Pub. Date: Sep. 10, 2009

(54) SYSTEM AND METHOD FOR PHOTOACOUSTIC IMAGING AND MONITORING OF LASER THERAPY

(75) Inventors: Xueding Wang, Canton, MI (US); David Chamberland, Medford, OR (US)

Correspondence Address: BROOKS KUSHMAN P.C., 1000 TOWN CENTER, TWENTY-SECOND FLOOR, SOUTHFIELD, MI, 48075, US

(73) Assignee: THE REGENTS OF THE UNIVERSITY OF MICHIGAN, Ann Arbor, MI (US)

(21) Appl. No.: 12/161,622

(22) PCT Filed: Jan. 19, 2007
(86) PCT No.: PCT/US2007/060764

§ 371 Date: Dec. 10, 2008

Related U.S. Application Data
(60) Provisional application No. 60/760,171, filed on Jan. 19, 2006.

(51) Int. Cl. A61B 18/20; A61B 8/00

(52) U.S. Cl. 606/10; 600/439

(57) ABSTRACT

A system and method for monitoring laser therapy of a target tissue include a therapeutic control unit having a first light source configured to deliver light to the target tissue for therapy, an ultrasonic transducer for receiving photoacoustic signals generated due to optical absorption of light energy by the target tissue, and a monitoring control unit in communication with the ultrasonic transducer for reconstructing photoacoustic tomographic images from the received photoacoustic signals to provide an optical energy deposition map of the target tissue. A second light source utilized for imaging may also be provided.

### CROSS-REFERENCE TO RELATED APPLICATIONS

This application claims the benefit of U.S. provisional application Ser. No. 60/760,171 filed Jan. 19, 2006 which is incorporated by reference herein.

### BACKGROUND OF THE INVENTION

1. Field of the Invention

This invention relates to photoacoustic imaging and monitoring of laser therapy.

2. Background Art

Intravenous and arterial occlusion is a commonly encountered vascular complication. Large, medium, and small sized vessels can become occluded for various reasons including both hereditary and acquired hypercoagulable states. Depending on the site of the occlusion, blood clot (e.g. plaque and thrombus) removal may be necessary.

In recent years, techniques involving fiber optics and laser ablation for therapy of strokes have been described and undergone human testing with hopes of providing rapid, safe, effective treatments. These interventions replace or are in addition to various pharmaceutical remedies used for stroke treatment, and are used target cerebral clots in the arteries of the brain. Similar techniques are also used for coronary artery disease, bypass grafts, femoral artery disorders, and peripheral vascular occlusion. An example includes laser thrombolysis, which is an interventional procedure for removing arterial plaque and thrombus (clots) by delivering laser pulses or continuous waves via an intravascular catheter. The removal of the clot results in a restoration of blood flow while maintaining vascular integrity. Examples of devices used for laser thrombolysis include excimer lasers and the LaTIS laser device (LaTIS, Inc., Coon Rapids, Minn.), which uses laser energy to ablate clots in arteries 2 to 5 mm in diameter, including thrombus within the internal carotid artery, M1 or M2 branches of the middle cerebral artery, and the anterior cerebral, vertebral, basilar, and posterior cerebral arteries.

In laser thrombolysis, when light is delivered into a vessel through an optical fiber or a light guide within a catheter, the light is absorbed by the thrombus (or plaque), vessel wall, and other surrounding tissues. The amount of energy absorbed by each of these components depends on the wavelength of the light. Previously, a continuous wave laser has been utilized to remove either arterial or venous obstructions; however, irradiation by such a laser does not confine the heat produced to the target area. The diffusion of heat out of the target area can result in thermal necrosis and even charring in the surrounding tissue.

Tissue ablation using ultrashort pulsed lasers can effectively limit thermal effects to adjacent tissues. The limiting pulse length is determined by the thermal relaxation time of the tissue, which is the time for heat to diffuse out of the irradiated volume and is determined by the thermal diffusivity of the tissue and the dimensions of the volume. When laser energy is deposited in pulses shorter than the thermal relaxation time, heat accumulates and high temperatures are achieved. Tissue ablation can then occur before the heat diffuses out of irradiated volume. This confinement of heat can reduce the thermal damage incurred by adjacent tissue. With short laser pulses, the absorption of light by the thrombus leads to explosive vaporization of the clot and the subsequent formation of vapor bubbles. The dynamics of these rapidly expanding and collapsing bubbles generate pressure transients which exert mechanical forces on the clot leading to the removal of more clot, which may be retrieved by a negative pressure intravascular catheter port.

Besides intravascular laser thrombolysis, laser therapy has been employed in the diagnosis and/or therapy of many diseases associated with many other organs in the human body including, but not limited to, the urinary, system, renal system, gastrointestinal system, pulmonary (including nose and mouth) system, female or male genital system, and auditory system. For example, use of laser ablation in endourology presents versatility and utility for multiple clinical applications, including fragmentation of stones, incision of ureteral and urethral structures, coagulation of bladder tumors, and enucleation of the prostate for benign hyperplasia.

While laser-based therapies have been developed and tried for years, problematic technical issues still exist. For laser thrombolysis or any other laser therapy, it is desirable to guide and optimize treatment efficacy by monitoring laser energy deposition within the target tissues and surrounding tissues in order to avoid side effects. In order to make laser thrombolysis a safe and rapid procedure so that it may be accepted as a standard treatment modality, technologies that can achieve quick (or even real-time) sensitive and accurate monitoring of the therapeutic procedure are greatly needed.

### BRIEF DESCRIPTION OF THE DRAWINGS

FIG. 1 is a schematic diagram depicting photoacoustic imaging and sensing of laser thrombolysis according to the present invention; and

FIG. 2 depicts an exemplary timing series for photoacoustic imaging and sensing of laser thrombolysis according to the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention.

The present invention includes a noninvasive imaging and sensing system and method employing photoacoustic imaging and laser-based catheters. Photoacoustic imaging and sensing of endovascular laser ablation therapy such as laser thrombolysis may provide an operating physician involved in thrombus or clot removal with a real time image and energy deposition map of the involved vessel at the site of the clot. This information, as well as the real-time blood flow that can also be monitored by the system of the present invention, are important parameters in evaluating the therapeutic procedural goals of techniques such as laser thrombolysis, enabling physicians to limit surrounding vessel and tissue damage. The real time information provided by the system and method according to the present invention may enable the operating physician to control and optimize the therapy efficiently, or even shut off the laser light automatically through a feedback system should unwanted damage start to happen.

Photoacoustic tomography (PAT) may be employed for imaging tissue structures and functional changes and describing the optical energy deposition in biological tissues with both high spatial resolution and high sensitivity. PAT employs optical signals to generate ultrasonic waves. In PAT, a short-pulsed electromagnetic source—such as a tunable pulsed laser source, pulsed radio frequency (RF) source or pulsed lamp—is used to irradiate a biological sample. The photoacoustic (ultrasonic) waves excited by thermoelastic expansion are then measured around the sample by high sensitive detection devices such as, but not limited to, ultrasonic transducer(s) made from piezoelectric materials and optical transducer(s) based on interferometry. Photoacoustic images are reconstructed from detected photoacoustic signals generated due to thermoelastic expansion occurring from the optical absorption in the sample through a reconstruction algorithm, where the intensity of photoacoustic signals is proportional to the optical energy deposition.

Optical signals, employed in PAT to generate ultrasonic waves in biological tissues, present high electromagnetic contrast between various tissues, and also enable highly sensitive detection and monitoring of tissue abnormalities. It has been shown that optical imaging is much more sensitive to detect early stage cancers than ultrasound imaging and X-ray computed tomography. The optical signals can present the molecular conformation of biological tissues and are related to significant physiologic parameters such as tissue oxygenation and hemoglobin concentration. Traditional optical imaging modalities suffer from low spatial resolution in imaging subsurface biological tissues due to the overwhelming scattering of light in tissues. In contrast, the spatial resolution of PAT is only diffraction-lirnited by the detected photoacoustic waves rather than by optical diffusion; consequently, the resolution of PAT is excellent (60 microns, adjustable with the bandwidth of detected photoacoustic signals). Besides the combination of high electromagnetic contrast and high ultrasonic resolution, the advantages of PAT also include good imaging depth, relatively low cost, non-invasive, and non-ionizing.

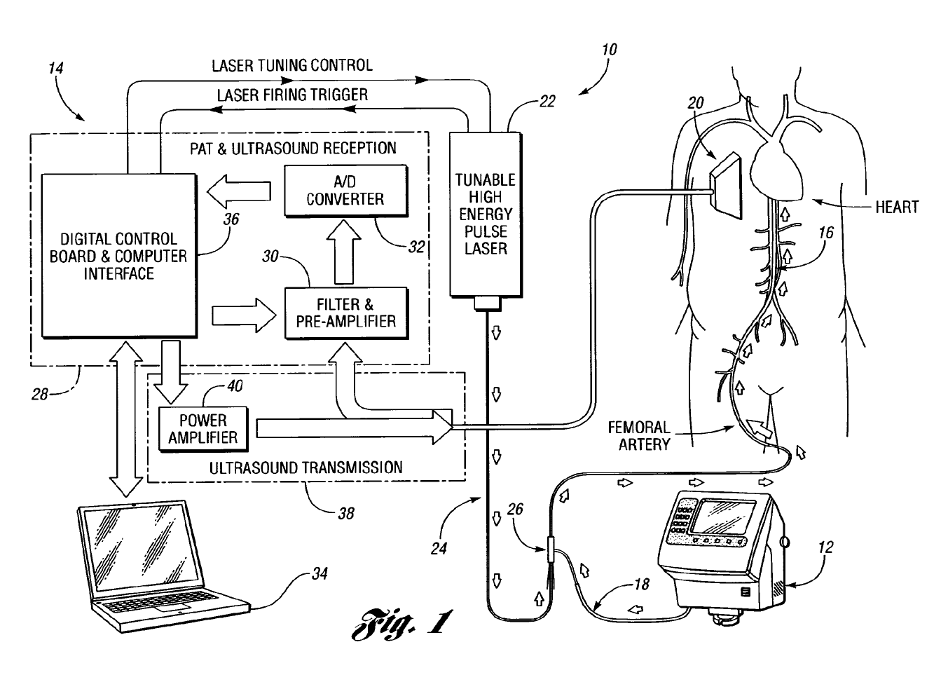

A therapeutic monitoring system according to the present invention is depicted in FIG. 1 and designated generally by reference numeral 10. System 10 may include a therapeutic control unit such as laser thrombolysis control unit 12, and a monitoring control unit such as photoacoustic and ultrasound system 14. Thrombolysis control unit 12 may include a light source for producing light energy in the form of light pulses or continuous waves which can be delivered to the site of the clot or other local or distant target tissue through a catheter 16 via optical fibers 18, a fluid core light guide, or the like. In one example, catheter 16 can be inserted into the femoral artery in the leg and advanced to an occlusion in the coronary artery as is known in the art. Of course, any catheter and target tissue location is fully contemplated in accordance with the present invention. Furthermore, it is understood that “target tissue” as used herein may refer to a clot and/or the surrounding vasculature or other tissues, as well as any other area of a living organism or non-living media.

If the light source used for therapy is a pulsed laser with short pulse duration, this light source may also enable photoacoustic imaging. In particular, when pulsed light is absorbed by the tissue in the clot, photoacoustic waves will be generated due to the optical absorption of biological tissues (i.e., optical energy deposition). Therefore, thrombolysis control unit 12 may generate laser pulses utilized for both thrombolytic and PAT purposes, wherein the light source provided by unit 12 may have a tunable wavelength. The photoacoustic signals may be detected external to the human body by a transducer 20, such as a high-sensitivity wide-bandwidth ultrasonic transducer, and used to reconstruct photoacoustic images using PAT. Transducer 20 can be any ultrasound detection device, e.g. single element transducers, 1D or 2D transducer arrays, optical transducers, transducers of commercial ultrasound machines, and others. The photoacoustic signals can be scanned along any surfaces around the target tissue. Moreover, detection at the detection points may occur at any suitable time relative to each other.

More particularly, the parameters of ultrasonic transducer 22 include element shape, element number, array geometry, array central frequency, detection bandwidth, sensitivity, and others. Transducers with designs such as, but not limited to, linear, arcuate, circular, and 2D arrays, can be applied for photoacoustic signal receiving, wherein the design of transducer 20 may be determined by the shape and location of the studied tissue, the expected spatial resolution and sensitivity, the imaging depth, and others. For example, for laser thrombolysis of peripheral vascular occlusion in the arms or legs, the transducer can be a circular shaped array around the arms or legs. In order to realize 3D imaging, the circular array may scan along the arms or legs. Also, a 2D transducer array with a cylindrical shape surface or a planar surface can be employed to achieve real-time imaging of the therapeutic procedure. As another example, for laser thrombolysis of cerebral blood vessels, a 2D transducer array with a semi-spherical shape to cover the skull may be utilized. In general, transducer 20 may include a 1D array that is able to achieve 2D imaging of the cross section in the tissue with single laser pulse. The imaging of a 3D volume in the tissue can be realized by scanning the array along its axis. In order to achieve 3D photoacoustic imaging at one wavelength with a single laser pulse, a 2D transducer array could instead be employed for signal detection.

Besides extra-vascular ultrasound detection, the photoacoustic signals generated by laser pulses according to the present invention can also be measured through an intravascular ultrasound technique. In this case, a small ultrasonic transducer (not shown) may be inserted into the vessel through the catheter together with an optical fiber (or light guide). The ultrasonic transducer may be positioned very close to the site of the clot and may scan the light-generated photoacoustic signals for imaging and sensing.

A continuous wave (CW) light or a laser with long pulse duration (e.g., on the order of microseconds) may incorporated in thrombolysis control unit 12 for therapeutic purposes. These kinds of light may not generate effective photoacoustic signals for photoacoustic imaging. Therefore, a separate PAT laser source can be utilized. As shown in FIG. 1, a light source, such as a high energy pulse laser 22 (e.g., Ti:Sapphire laser, optical parametric oscillator (OPO) system, dye laser, and arc lamp), may be provided to deliver light pulses to the clot. In general, laser 22 may provide pulses with a duration on the order of nanoseconds (e.g., 5 ns) and a narrow linewidth on the order of nanometers for irradiating the site of the clot. The wavelength of laser 22 may be tunable over a broad region (e.g., from 300 nm to 1850 nm), but is not limited to any specific range. The selection of the laser spectrum region depends on the imaging purpose, specifically the biochemical substances to be studied. However, in order to direct therapy by describing the light energy distribution in tissues, the wavelength for PAT should be the same as that used in laser therapy. Laser 22 may be connected to an optical fiber bundle 24 or the like which may deliver laser light to the target tissue via coupling into catheter 16 using a Y-shaped optical coupler 26 or other means, such that the light from unit 12 and laser 22 may be delivered to the same location in the tissue.

The received photoacoustic signals may be processed by reception circuitry 28, optionally including a filter and pre-amplifier 30 and an A/D converter 32, and collected by a computer 34 through a digital control board and computer interface 36. Digital control board and computer interface 36 may also receive the triggers from laser 22. At the same time, computer 34 may also control the tuning of the wavelength of laser 22 through digital control board and computer interface 36. A “computer” may refer to any suitable device operable to execute instructions and manipulate data, for example, a personal computer, work station, network computer, personal digital assistant, one or more microprocessors within these or other devices, or any other suitable processing device. It is understood that reception circuitry 28 shown in FIG. 1 is only an example, and that other circuitries with similar functions may also be employed in system 10 according to the present invention for control and signal receiving.

The detected photoacoustic signals can be processed by computer 34 and utilized for 3D image reconstruction utilizing PAT. Photoacoustic tomographic images presenting the tissue structures and abnormalities and a map of the optical energy deposition of the intra- and extra-vascular space around the clot may be generated with both high spatial and temporal resolution through any basic or advanced reconstruction algorithms based on diffusing theory, back-projection, filtered back-projection, and others. The reconstruction of optical images may be performed in both the spatial domain and frequency domain. PAT produces a real time image and overlying energy map for the operating physician to guide the amount of applied energy focused on the clot or plaque so as to maximize essential removal while preserving surrounding vessel wall and extravascular tissue. Therefore, with the system and method of the present invention, the physician may be provided with a real time evaluation of tissue responses to therapy, such that the treatment plan may be adjusted on-line. Before or after the generation of photoacoustic, optical and ultrasound images, any signal processing methods can be applied to improve the imaging quality. Photoacoustic images may be displayed on computer 34 or another display.

As described above, pulsed light from light source 22 can induce photoacoustic signals in the clot that are detected by ultrasonic transducer 20 to generate 2D or 3D photoacoustic tomographic images of the clot and surrounding tissues. By varying the light wavelength in the tunable region and applying laser pulses at two or more wavelengths to the tissue, the local spectroscopic absorption of each point in the target tissue can be generated and analyzed using computer 30. The photoacoustic image presents the optical absorption distribution in biological tissues, while spectroscopic photoacoustic data reveal not only the morphological information but also functional biochemical information in biological tissues. Spectroscopic photoacoustic tomography (SPAT) may yield high resolution images and point-by-point spectral curves for substance identification within a three-dimensional specimen, such as biological organs.

At each voxel in a three dimensional area, a spectroscopic curve indicating the concentration of various absorbing materials can be produced. The subsequent mapped point-by-point spectroscopic curves of the obtained tissue image can describe spatially distributed biological and biochemical substances including, but not limited to, intrinsic biological parameters such as glucose, hemoglobin, cytochromes, blood concentration, water concentration, and lipid concentration along with functional parameters such as oxygen saturation. These parameters are useful in evaluating any damage in surrounding tissues (e.g. vessel wall) caused by the thrombolytic laser pulses. Extrinsic entities including, but not limited to, molecular or cellular probes, markers, antibodies, or pharmaceutical or contrast agents added for any therapeutic or diagnostic reason including image enhancement, clot ablation, or refined molecular or cellular mapping could also be incorporated in the system and method described herein.

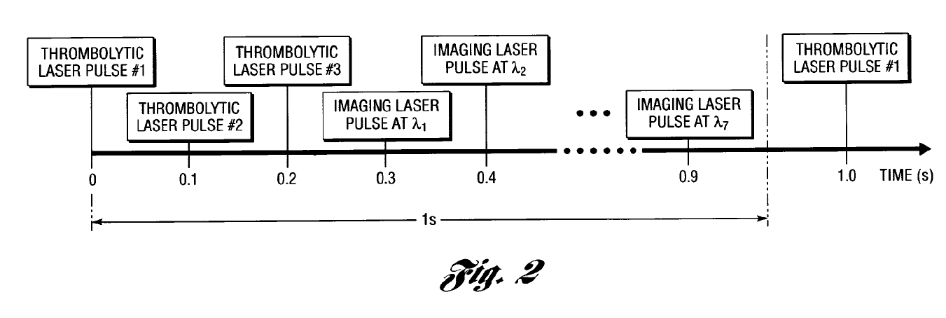

With reference now to FIG. 2, laser pulses at wavelengths for sensing and enabling image and spectroscopic data acquisition can be interspersed with thrombolytic laser pulses. In the example shown herein, in a 1 second period, 3 thrombolytic laser pulses for therapy followed by 7 imaging laser pulses at different wavelengths for SPAT can be sent. After that, the next 3 thrombolytic pulses and then 7 imaging pulses may be delivered in the subsequent 1 second period. Of course, other designs of timing series may also be applied, depending on the purpose of the SPAT and requirement of the therapy. For example, 1 therapeutic thrombolytic laser pulse followed by 4 imaging laser pulses at different wavelengths for SPAT can be sent. In this case, the total time for one period may be 0.5 second.

Referring again to FIG. 1, reception circuitry 28 may be utilized both for PAT and for ultrasound signal receiving and processing. By using ultrasonic transducer 20 as both a transmitter and receiver of signals, ultrasound signal transmission may also be achieved through an ultrasound transmission system 38 in communication with digital control board and computer interface 36. Ultrasound transmission system 38 is capable of generating high voltage pulses and corresponding delays for each transducer element, and may include an amplifier 40. A pulse-echo technique may be used for the pure ultrasound imaging. The whole transducer array or overlapping sub arrays can be used to transmit and receive ultrasound pulses and then generate ultrasound images of the target tissue through the technique of synthetic aperture. Multiple transmissions can be used for each subarray position in order to create multiple focal zones and thereby achieve uniform illumination along the propagation path. System 10 according to the present invention can realize not only gray scale ultrasound images to present tissue morphology in 2D or 3D space, but also Doppler ultrasound images to depict real-time blood flow in biological tissues and provide another assessment of the therapeutic effect. The photoacoustic and ultrasound imaging results of the same target tissue may be combined together through image registration and used to provide very comprehensive diagnostic information.

The system and method according to the present invention may further provide an objective map of adequate clot or other target tissue removal, which likely would inhibit chances of recurrence, and can document the amount of consequent restored blood flow to return pre-occlusion local and regional vascular hemodynamics. A negative pressure port may not be able to retrieve 100% of a clot, causing that portion of the clot which is not retrieved to disseminate to the tissues, likely causing microinfarction. Using PAT and possibly Doppler ultrasound according to the present invention, the system and method described herein may alert the physician as to when blood flow is resuming such that the negative pressure port can be readied or its function modified (e.g., increasing the negative pressure, extending a device to catch broken-off portions of the clot, etc.).

In accordance with the present invention, the PAT and ultrasound reception and the ultrasound transmission in FIG. 1 can be realized with any proper design of circuitry 28, 38. The circuitry performs as an interface between the computer and transducer 20, laser 22, and other devices. “Interface” may refer to any suitable structure of a device operable to receive signal input, send control output, perform suitable processing of the input or output or both, or any combination of the preceding, and may comprise one or more ports, conversion software, or both. A component of a reception system may comprise any suitable interface, logic, processor, memory, or any combination of the preceding.

According to another aspect of the present invention, computer 34, PAT and ultrasound reception circuitry 28 and ultrasound transmission circuitry 38 can be integrated with laser thrombolysis control unit 12. Through such an integrated control unit, both control and monitoring of the therapeutic procedure may be achieved. The integrated control unit may generate and analyze point-by-point imaging and spectroscopic information of tissues under laser ablation therapy. Through programming, if unwanted damage starts to happen, this control unit may shut off the laser light automatically through a feedback system.

The spatial resolution of PAT is determined by the frequency bandwidth of ultrasonic transducer 20 and the pulse duration of laser 22. Using a 10 MHZ transducer array with a 100% bandwidth and laser pulses with 10 ns pulse duration, the highest achievable spatial resolution may be about 160 micrometers. When laser pulses with longer duration are employed for therapy, the spatial resolution may be degraded. For example, using a 10 MHZ transducer array with a 100% bandwidth and laser pulses with 100 ns pulse duration, the highest achievable spatial resolution may be about 300 micrometers.

Advantages of the system and method of the present invention may include the ability to monitor in real time optical energy deposition, tissue structural and functional changes, along with blood flow at the site of clot removal, thereby limiting the damage to vascular and extravascular tissue. Spectroscopic information can also be obtained on a point-by-point basis in the three dimensional tissue, which may present biological and biochemical changes in vessel walls and surrounding tissues with great sensitivity during the laser ablation therapy. This enables the study of a target tissue using both morphological and spectroscopic information with high spatial resolution and high sensitivity. According to the present invention, the monitoring of laser ablation therapy can be done in a non-invasive, non-intrusive manner without using an ionizing source and incorporated relatively easily with existing medical instrumentation. Extrinsic substances can be added for further spectroscopic or other characterization of intrinsic biological substances or parameters.

Real time, accurate energy deposition maps in the clotted vessels may be provided, which minimizes risk of vessel wall or extravascular tissue iatrogenic trauma. The functional imaging ability provided by the system and method of the present invention may be sensitive not only to different soft tissues that have different electromagnetic properties, but also to functional changes in biological tissues. The molecular and cellular imaging ability provided by the spectroscopic information which may be obtained by the system of the present invention manifests the presence, concentrations, and changes of the biological and biochemical substances in the localized areas in the specimen with both high sensitivity and high specificity. Furthermore, the ultrasonic transducer employed in the system according to the present invention can enable real time monitoring of blood flow. Still further, the imaging and sensing system described herein may cost much less and be a more mobile system than MRI.

The system and method according to the present invention can be used in imaging and sensing of all types of laser therapy based on pulsed light. Besides intravascular laser thrombolysis, the system of the present invention could also be employed in any case where light is delivered within the body including, but not limited to, the urinary system, renal system, gastrointestinal system, pulmonary (including nose and mouth) system, female or male genital system, and auditory system, for diagnostic or therapeutic reasons. For example, use of laser ablation in endourology presents versatility and utility for multiple clinical applications, including fragmentation of stones, incision of ureteral and urethral strictures, coagulation of bladder tumors, and enucleation of the prostate for benign hyperplasia. The photoacoustic technology described herein may aid imaging and sensing of laser therapy of such disorders. As another example, laser ablation of endometriosis allows for treatment of small affected areas with minimal impact on the surrounding tissues, where photoacoustic imaging and sensing technology of the present invention can also be applied. Spectroscopic identification of general neoplasia and more specifically certain types of cancer potentially could also be realized in real time. Embodiments of the present invention could include adding imaging and spectroscopic sensing to any type of endoscopic probe such as those used in colonoscopy, upper GI endoscopy, nasopharyngoscopy, bronchoscopy, cystoscopy, and laproscopy.

The addition of extrinsic agents such as pharmaceutical substances or imaging contrast agents could be used for image, spectroscopic data, or therapeutic enhancement in accordance with the present invention. The system and method according to the present invention could also be used for point to point treatment, i.e. once a characteristic spectral curve is detected at any three-dimensional location within the sample, thermal or photo or acoustic signals could be directed to that location for therapies needing thermal ablation or photoactivation of a pharmaceutical compound.

Monitoring can take place in any living organism, including animals and humans, and be used to evaluate any vascular disease in real time, including atherosclerosis or vasculopathy associated with scieroderma. Furthermore, spectroscopic data on the development of associated lesions may be provided continuously over time, leading to further understanding of pathogenesis along with monitoring vascular effects of medications, such as statins. The system and method according to the present invention could also be used in any non-living media, including industrial settings such as CNC machining workstations where the object of interest is favorable to optical signal producing thermoelastic expansion causing acoustic wave propagation. Such image and spectroscopic data could be used in machining feedback monitoring systems. The system and method according to the present invention could also be used in the transfer or refining process of natural resources such as oil, where real time imaging and point by point spectroscopic data along with flow rate could be produced for monitoring of conditions such as impurities.

The system and method of the present invention include the ability to provide real time sensing, including image, spectroscopic, and flow data acquisition in both medical and industrial applications. Intrinsic speciren-acquired data characteristics could be enhanced by extrinsic substances in any application. The monitoring of laser therapy can be done in a non-invasive, non-intrusive manner without using an ionizing source. The system and method according to the present invention, uses photoacoustic techniques to realize quick (real time) accurate and sensitive monitoring of the therapeutic procedure without requiring changes to existing laser systems.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for monitoring laser therapy of a target tissue, the system comprising:
a therapeutic control unit having a first light source configured to deliver light to the target tissue for therapy;
an ultrasonic transducer for receiving photoacoustic signals generated due to optical absorption of light energy by the target tissue; and
a monitoring control unit in communication with the ultrasonic transducer for reconstructing photoacoustic tomographic images from the received photoacoustic signals to provide an optical energy deposition map of the target tissue.

2. The system according to claim 1, further comprising a second light source in communication with the monitoring control unit, the second light source comprising a laser configured to deliver short duration light pulses to the target tissue for imaging.

3. The system according to claim 2, wherein the first light source and the second light source operate at the same wavelength.

4. The system according to claim 2, further comprising a catheter in communication with the first light source for delivering light to the target tissue, wherein the second light source is coupled to the catheter using a Y-shaped optical coupler.

5. The system according to claim 2, wherein the second light source has a tunable wavelength.

6. The system according to claim 5, wherein upon the delivery of light pulses of two or more different wavelengths to the target tissue, the monitoring control unit is configured to determine the local spectroscopic absorption of substances at any location in the target tissue.

7. The system according to claim 6, wherein the substances include intrinsic or extrinsic substances.

8. The system according to claim 2, wherein the monitoring control unit receives a firing trigger from the second light source.

9. The system according to claim 2, wherein the monitoring control unit controls tuning the wavelength of the second light source.

10. The system according to claim 1, wherein the therapeutic control unit and the monitoring control unit are integrated into the same system.

11. The system according to claim 1, wherein the monitoring control unit is in communication with the first light source and is configured to shut off the first light source automatically through a feedback system.

12. The system according to claim 1, wherein the ultrasonic transducer is configured to transmit ultrasound signals to the target tissue for generating at least one of ultrasound images and Doppler ultrasound images.

13. The system according to claim 1, wherein the monitoring control unit is configured to combine images of the target tissue through image registration.

14. A system for monitoring laser therapy of a target tissue, the system comprising:
a therapeutic control unit having a first light source configured to deliver light to the target tissue for therapy;
a second light source including a laser configured to deliver short duration light pulses to the target tissue for imaging;
an ultrasonic transducer for receiving photoacoustic signals generated due to optical absorption of light energy by the target tissue; and
a monitoring control unit in communication with the second light source and the ultrasonic transducer for reconstructing photoacoustic tomographic images from the received photoacoustic signals to provide an optical energy deposition map of the target tissue.

15. A method for monitoring laser therapy of a target tissue, comprising;
providing a first light source for delivering light to the target tissue for therapy;
receiving photoacoustic signals generated due to optical absorption of light energy by the target tissue with an ultrasonic transducer;
reconstructing photoacoustic tomographic images from the received photoacoustic signals to provide an optical energy deposition map of the target tissue.

16. The method according to claim 15, further comprising providing a second light source comprising a laser for delivering short duration light pulses to the target tissue for imaging.

17. The method according to claim 16, wherein the second light source has a tunable wavelength for delivering light pulses of two or more different wavelengths to the target tissue.

18. The method according to claim 17, further comprising determining the local spectroscopic absorption of substances at any location in the target tissue.

19. The method according to claim 18, further comprising directing therapeutic signals to the location within the target tissue.

20. The method according to claim 16, further comprising operating the first light source and the second light source at the same wavelength.

21. The method according to claim 16, further comprising interspersing light pulses from the first light source with light pulses from the second light source.

22. The method according to claim 15, further comprising transmitting ultrasound signals to the target tissue for generating at least one of ultrasound images and Doppler ultrasound images.

23. The method according to claim 15, further comprising combining images of the target tissue through image registration.
